SEQUENCE LISTING (4 sequences)

SEQ ID NO: 1 - DNA, 16 bp, Artificial Sequence
  Tail Sequence
aaaaaaaaaaaaaaaa

SEQ ID NO: 2 - DNA, 16 bp, Artificial Sequence
  Tail Sequence
tttttttttgccggcc

SEQ ID NO: 3 - DNA, 16 bp, Artificial Sequence
  Tail Sequence
aaaaaaaaaccccccc

SEQ ID NO: 4 - DNA, 16 bp, Artificial Sequence
  Tail Sequence
aaaaaaaaacggccgg

(19) United States
(12) Patent Application Publication
(10) Pub. No.: US 2005/0175993 A1
(43) Pub. Date: Aug. 11, 2005

(54) Method for making full-length coding sequence cDNA libraries

(76) Inventor: Chia-Lin Wei, Vacaville, CA (US)

Correspondence Address: Barry Swanson;Swanson & Bratschun, 1745 Shea Center Drive, Suite 330, Highlands Ranch, CO, 80129, US

(21) Appl. No.: 10/121,641

(22) Filed: Apr. 12, 2002

(51) Int. Cl. C12Q 1/68; C12P 19/34

(52) U.S. Cl. 435/6; 435/91.2

(57) ABSTRACT

The present invention relates to a method for making cDNA libraries wherein the cDNA inserts comprise the full-length of the coding sequences but having lengths less than the full-length of the mRNA. The method comprises binding a tag molecule to a diol structure present in the 5′ Cap sites of mRNAs, forming RNA-DNA hybrids by reverse transcription to synthesize the first cDNA strand, separating RNA-DNA hybrids carrying a DNA corresponding to a full-length of mRNAs from RNA-DNA hybrids formed above by using a function of the tag molecule, and synthesizing the second cDNA strand by self-priming the first cDNA strand. The resulting cDNA libraries do not contain the full-length of the mRNAs but do contain the full-length of the coding sequences of the mRNAs.

### FIELD OF THE INVENTION

The present invention relates to a method for making full-length cDNA libraries wherein the cDNA inserts comprise the full-length of the coding sequences but having lengths less than the full-length of the mRNA. Specifically, it relates to a method for making full-length coding sequence cDNA libraries by a method for purification of full-length coding sequence cDNAs utilizing chemical modification of mRNAs.

### BACKGROUND OF THE INVENTION

Methods for synthesizing cDNAs are essential techniques for researches in the fields of medical science and biology as an indispensable method for analyzing gene transcripts. Any DNA genetic information manifests physiological activity through transcripts and a potential means for analyzing such transcripts is cDNA cloning. In cDNA syntheses according to conventional methods, clones are ultimately isolated from a cDNA library prepared from poly A sites by using oligo dT as a primer.

Conventional methods for synthesizing cDNAs have, for example, the following problems:
1. cDNAs covering most part of transcripts can be obtained by using a random primer. However, those cDNAs are short fragments and clones covering the entire coding regions cannot be isolated.
2. Any cDNA obtained by using oligo dT as a primer contains 3′ end. However, due to the secondary structure of the mRNA and processitivity of the reverse transcriptase, the reverse transcriptase cannot reach the 5′ Cap site, the 5′ upstream should be further isolated and analyzed by the primer elongation method and 5′RACE or the like.
3. Efficiency of any conventional methods for isolating cDNAs in their full-lengths including those methods mentioned above is not sufficient (only 2,000,000 recombinant phages can be obtained from 100 μg of mRNA). Therefore, more efficient techniques are desired for practical purposes.

As conventional methods for synthesizing full-length cDNAs, the following methods can be mentioned; the method utilizing a Cap binding protein of yeast or Hela cells for labeling the 5′ Cap site (I. Edery, et al., “An Efficient Strategy To Isolate Full-length cDNAs Based on an mRNA Cap Retention Procedure (CAPture)”, Mol. Cell. Biol. 15:3363-71, 1995); the method where phosphates of incomplete cDNAs without 5′ Cap are removed by using alkaline phosphatase and then the whole cDNAs are treated with de-capping enzyme of tobacco mosaic virus so that only the full-length cDNAs have phosphates (K. Maruyama, et al., “Oligo-capping: a simple method to replace the cap structure of eukaryotic mRNAs with oligoribonucleotides”, Gene 138:171-4, 1995; S. Kato, et al., “Construction of a human full-length cDNA bank”, Gene 150:243-50, 1995) and the like.

In some cases, when using a conventional cDNA library, it is essential for analysis of gene structures in their full-lengths to synthesize 5′ upstream regions by the primer elongation method, or perform gene working of the 5′ upstream regions by cDNA synthesis using a random primer. In other cases, it is the coding sequences of the mRNA substrate that is crucial or desired and thus it is not necessary to synthesize the entire 5′ upstream region.

Therefore, an object of the present invention is to provide a novel method in which 5′ Cap site can be more efficiently labeled compared with the labeling by the proteins reactions such as those by the conventional recombinant Cap binding protein and the de-capping enzyme of tobacco mosaic virus.

Another object of the present invention is to provide a method for making full-length coding sequence cDNA libraries utilizing the novel method of the present invention for labeling of the 5′ Cap site.

U.S. Pat. Nos. 6,143,528 and 6,174,669 disclose a method for making full-length cDNA libraries, which is for making libraries of cDNAs having a length corresponding to a full-length of mRNAs and comprises the following steps of; binding a tag molecule to a diol structure present in 5′ Cap (⁷Me Gₚₚₚ N) sites of mRNAs, forming RNA-DNA hybrids by reverse transcription using primers such as oligo dT and the mRNAs connected with the tag molecule as templates, separating RNA-DNA hybrids carrying a DNA corresponding to a full-length of mRNAs from the RNA-DNA hybrids formed above by using function of the tag molecule, and synthesizing the second DNA strand by binding a G tail addition by terminal deoxynucleotidyl transferase followed by priming with oligo C. However, these disclosures do not disclose a method for making a full-length coding sequence cDNA having lengths less than the full-length of the mRNA. Also, the methods disclosed by these disclosures do not synthesize the second strand of the cDNA using the self-priming of the first strand of the cDNA, or a method does not rely on the addition of a poly-G tail by terminal deoxynucleotidyl transferase.

These disclosed methods are not efficient for constructing cDNA libraries containing the coding sequences. These methods have at least the following problems:
1. The dG (or A, T, or C) tail addition by terminal deoxynucleotidyl transferease (TDT) introduces artificial nucleotide sequences in the very 5′ end of the cDNA. Those sequences do not correspond to the authentic mRNA sequences produced inside the cells. The method of the present invention bypasses this procedure.
2. TDT incorporates different amounts of dG (or A, T, or C) into the 5′ end of the cDNA, so every cDNA molecule carries different sizes of oligomer stretch. The length can vary from 11 to 60 nucleotides. The difference in length can create different stability when the first strand cDNA is primed with oligo dC to synthesize the second strand cDNA. The shorter the dC stretch, the less stable is the priming. To increase the priming efficiency, these methods call for 35° C. annealing for second strand synthesis. In some cases, this temperature will cause the primer to misprime internally at DG-rich cDNA sequences.
3. These methods give poor sequencing efficiency. The sequences immediately following the GC-rich stretch usually failed to read.
4. Long GC-rich sequences at the 5′ untranslated terminal region will inhibit protein expression level from the cDNA clones because of the large degree of secondary structure.
5. There is no restriction enzyme site introduced between the authentic cDNA sequences and artificial GC stretch in these methods. If one wants to further subclone the authentic cDNA sequences from the GC sequences to another cloning vector, he has to use PCR amplification with gene specific primers. PCR tends to incorporate mismatch and, with gene specific primers, it is hard to scale to high throughput format.

The present invention is able to overcome the above mentioned problems.

### SUMMARY OF THE INVENTION

This present invention provides for a method for making a full-length coding sequence cDNA library, wherein said full-length coding sequence cDNA library is a library of cDNAs comprising the full-length of the coding sequences and having lengths less than the full-length of mRNAs.

This present invention further provides for a method for making a full-length coding sequence cDNA library, comprising: (a) forming RNA-DNA hybrids by reverse transcription starting from primers using mRNAs as templates; (b) binding a tag molecule to a diol structure present in the 5′ cap site of a mRNA forming a RNA-DNA hybrid; and (c) separating RNA-DNA hybrids carrying a DNA corresponding to a full-length mRNA from the RNA-DNA hybrids formed above by binding the tag molecule, wherein said DNA corresponding to a full-length mRNA are first cDNA strands; wherein said full-length coding sequence cDNA library is a library of cDNAs comprising the full-length of the coding sequences and having lengths less than the full-length of mRNAs.

The present invention also provides for a method for constructing a full-length coding sequence cDNA library, comprising: (a) binding a tag molecule to a diol structure present in 5′ cap sites of mRNAs by oxidizing the 5′ cap site diol to form a dialdehyde and reacting the resulting dialdehyde with a tag molecule having a group reactive with the dialdehyde; (b) forming RNA-DNA hybrids by reverse transcription using primers and the mRNAs binding the tag molecule as templates; and (c) separating RNA-DNA hybrids carrying a DNA corresponding to a full-length of mRNA from the RNA-DNA hybrids formed above by using a function of the tag molecule, wherein said DNA corresponding to a full-length mRNA are first cDNA strands; wherein said full-length coding sequence cDNA library is a library of cDNAs comprising the full-length of the coding sequences and having lengths less than the full-length of mRNAs.

The present invention further provides a method for making a full-length cDNA library, comprising: (a) binding a biotin molecule to a diol structure present in 5′ cap sites of mRNAs by oxidizing the 5′ cap site diol to form a dialdehyde and reacting the resulting dialdehyde with a biotin molecule having a group reactive with the dialdehyde; (b) forming RNA-DNA hybrids by reverse transcription using primers and the mRNAs bound to biotin molecules as templates; (c) digesting the RNA-DNA hybrids with an RNase capable of cleaving single strand RNA to cleave the single strand RNA parts of the hybrids carrying a DNA not corresponding to a full-length mRNA to remove biotin molecules from the hybrids, wherein said DNA not corresponding to a full-length mRNA is a first cDNA strand; and (d) separating RNA-DNA hybrids carrying a DNA corresponding to a full-length mRNA and binding the biotin molecules by (1) allowing them to react with avidin fixed on a solid support or (2) affinity chromatography to a solid support; wherein said full-length coding sequence cDNA library is a library of cDNAs comprising the full-length of the coding sequences and having lengths less than the full-length of mRNAs.

The present invention further provides a method for making a full-length cDNA library, comprising: (a) binding an avidin molecule to a diol structure present in 5′ cap sites of mRNAs by oxidizing the 5′ cap site diol to form a dialdehyde and reacting the resulting dialdehyde with an avidin molecule having a group reactive with the dialdehyde; (b) forming RNA-DNA hybrids by reverse transcription using primers and the mRNAs bound to avidin molecules as templates; (c) digesting the RNA-DNA hybrids with an RNase capable of cleaving single strand RNA to cleave the single strand RNA parts of the hybrids carrying a DNA not corresponding to full-length mRNAs to remove avidin molecules from the hybrids, wherein said DNA not corresponding to a full-length mRNA is a first cDNA strand; and (d) separating RNA-DNA hybrids carrying a DNA corresponding to a full-length mRNA and binding avidin molecules by (1) allowing them to react with biotin fixed on a solid support or (2) affinity chromatography to a solid support; wherein said full-length coding sequence cDNA library is a library of cDNAs comprising the full-length of the coding sequences and having lengths less than the full-length of mRNAs.

Preferably, the method further comprising the step of synthesizing second cDNA strands using as templates said first cDNA strands, wherein ligating an RNA or DNA oligomer to the 3′ end of said first cDNA strands is not required. Preferably, said synthesizing comprises self-priming said first cDNA strand. Preferably, said synthesizing and said digesting at least overlap. Preferably, the primer is oligo dT. Preferably, the RNase capable of cleaving single strand RNA is ribonuclease I.

### BRIEF DESCRIPTION OF DRAWINGS

FIG. 1 depicts a structure of mRNA having diol structures at both ends (the 5′ Cap site and 3′ site.

FIG. 2 depicts a reaction scheme representing oxidation of the diol structure of the 5″ Cap site of mRNA and addition of biotin hydrazide hereto.

FIG. 3 depicts a scheme showing each step of the method for making full-length coding sequence cDNAs (but not up to the 5′ end of the mRNA). The hatched bars indicate RNA, and the open bars indicate DNA. The black bars indicate linkers. The small circles labeled indicate biotin. The large circles indicate avidin beads.

### DESCRIPTION OF THE INVENTION

The coding sequences are the residues that encode amino acids, which are translated in the cell from which the mRNA is derived or obtained. The coding sequences correspond to the open reading frame or the intron or introns of a gene. The coding sequences are not found at the very 5′ end of each mRNA. The 5′ end of each mRNA contains a leader sequence of the mRNA. The object of the invention is to construct a cDNA library comprising the full-length of the coding sequences, but not the full-length of the entire mRNA. This is because the object of the invention is to facilitate the further investigation the full-length coding sequences, and the peptides they encode, but not the leader sequences of the mRNA of each gene encoding the peptides.

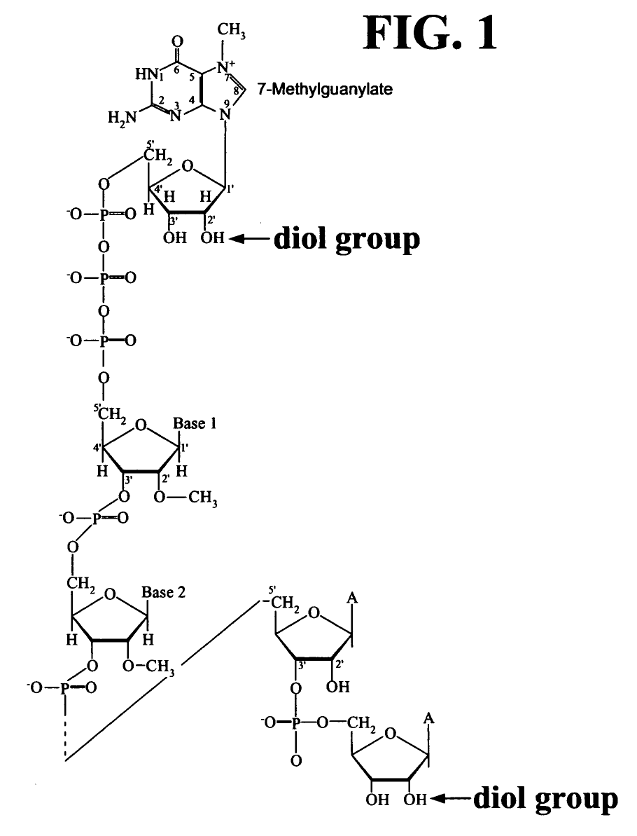

According to the method of the present invention, 5′ Cap site is labeled by chemical synthesis utilizing the structure specific for the 5′ Cap site, the diol structure, in order to enhance the recognition of the 5′ Cap site and to increase efficiency of the selection of full-length cDNAs (RNAs) (see FIG. 1).

That is, according to the method of the present invention, a tag molecule is first bound to a diol structure present in 5′ Cap (or “^{7Me}GₚₚₚN”) site of mRNAs. This tag molecule is chemically bound to the 5′ Cap site, and full-length cDNAs are synthesized by using mRNAs labeled with the tag molecule as a template to produce full-length cDNA library.

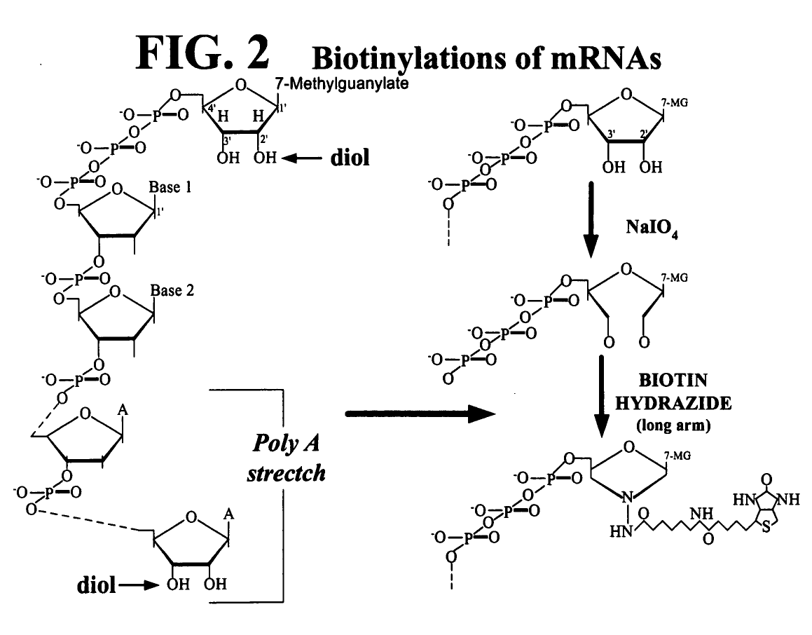

The binding of the tag molecule to the 5′ Cap site can be obtained by, e.g., oxidation ring-opening reaction of the 5′ Cap site diol structure with an oxidizing agent such as sodium periodate (NaIO₄) to form a dialdehyde and subsequent reaction of the dialdehyde with a tag molecule having a hydrazine terminus (see FIG. 2).

As the tag molecule having a hydrazine terminus, e.g., biotin molecule or avidin molecule having a hydrazine terminus can be mentioned. A molecule showing reaction specificity such as antigens or antibodies can also be used as the tag molecule. That is, the specific label used as the tag molecule is not particularly limited.

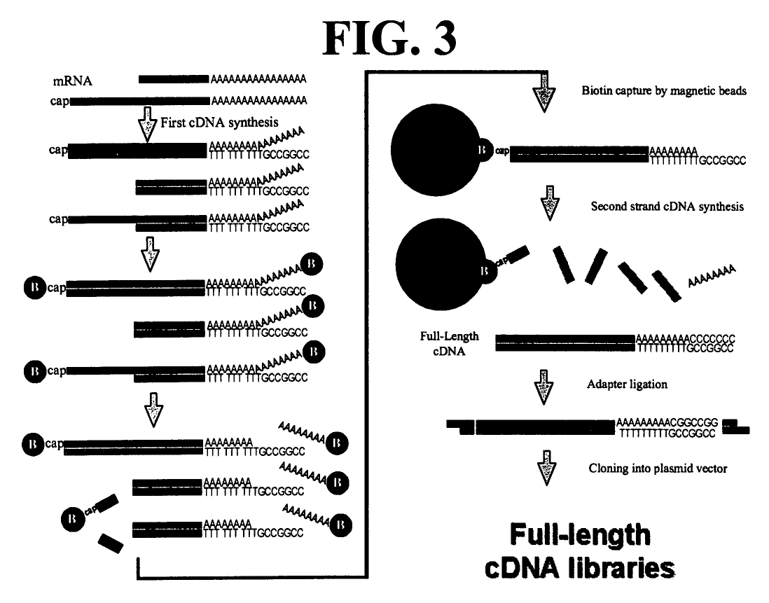

Exemplary process steps including (1) binding of tag molecule to (8) cloning of full-length cDNAs (tag molecule: biotin) are shown in FIG. 3.
(1). Biotinylation of diol groups
(2). Preparation of first cDNA strand
(3). Ribonuclease I (RNase I) Digestion
(4). Capture of full-length cDNA hybrids (with avidin beads)
(5). RNase H digestion (removal from the avidin beads)
(6). Preparation of second strand using DNA polymerase, DNA ligase, and dATPs, dCTPs, dGTPs, and dTTPs.
(7). Addition of linkers.
(8). Cloning into a vector.

The RNA-DNA hybrids can be produced by reverse transcription starting from a primer such as oligo dT using the mRNAs labeled with the bound tag molecule as a template. This production of RNA-DNA hybrids by reverse transcription utilizing a primer such as oligo dT can be performed by a conventional method. Either of steps (1) or (2) can be carried first, or both concurrently.

Further, RNA-DNA hybrids carrying a DNA corresponding to a full-length of mRNAs are separated from the whole RNA-DNA hybrids by using function of the tag molecule.

Specifically, the tag molecule is removed from those RNA-DNA hybrids carrying a DNA not corresponding to a full-length of mRNAs by digesting the hybrids with an RNase capable of cleaving single strand RNA to cleave the single strand parts of the hybrids. Then, those hybrids carrying a DNA corresponding to a full-length of mRNAs (full-length cDNAs extended to 5′ Cap) are separated by utilizing the function of the tag molecule.

For example, when the tag molecule is biotin molecule, hybrids carrying a DNA corresponding to a full-length of mRNAs can be separated by allowing the biotin molecules possessed by the RNA-DNA hybrids as the tag molecule to react with avidin fixed on a solid support. When the tag molecule is avidin molecule, hybrids carrying a DNA corresponding to a full-length of mRNA can be separated by allowing the avidin molecules possessed by the RNA-DNA hybrids as the tag molecule to react with biotin fixed on a solid support.

Therefore, one embodiment of the present invention relates to a method for making full-length coding sequence cDNA libraries, which is for making libraries of cDNAs having a length corresponding to a full-length coding sequence of mRNAs and comprises the following steps of;
binding a biotin molecule to a diol structure present in 5′ Cap (^{7Me}GₚₚₚN) site lo of mRNAs,
forming RNA-DNA hybrids by reverse transcription using primers and the mRNAs connected with biotin molecule as templates,
digesting the formed hybrids with an RNase capable of cleaving single strand RNA to cleave the single strand RNA parts of the hybrids to remove biotin molecules from the hybrids, and
separating RNA-DNA hybrids carrying a DNA corresponding to a full-length of mRNAs and binding the biotin molecules by allowing them to react with avidin fixed on a solid support.

Another embodiment of the present invention relates to a method for making full-length coding sequence cDNA libraries, which is for making libraries of cDNAs having a length corresponding to a full-length coding sequence of mRNAs and comprises the following steps of;
binding an avidin molecule to a diol structure present in 5′ Cap (^{7Me}GₚₚₚN) site of mRNAs,
forming RNA-DNA hybrids by reverse transcription using primers and the mRNAs connected with the avidin molecule as templates,
digesting the formed hybrids with an RNase capable of cleaving single strand RNA to cleave the single strand RNA parts of the hybrids to remove avidin molecules from the hybrids, and
separating RNA-DNA hybrids carrying a DNA corresponding to a full-length of mRNAs and binding avidin molecules by allowing them to react with biotin fixed on a solid support.

As the RNase capable of cleaving single strand RNA, e.g., ribonuclease I can be mentioned. Selection of the hybrids carrying a DNA corresponding to a full-length of mRNA from the whole RNA-DNA hybrids can be performed by any means other than those using an enzyme capable of cleaving single strand RNA. That is, the method for selecting the hybrids is not particularly limited.

According to the method of the present invention, cDNAs are further collected from the separated hybrids carrying DNAs corresponding to full-lengths of mRNAs. The collection of the cDNAs can be performed by, for example, treating the separated hybrids carrying DNAs corresponding to full-lengths of mRNAs with alkaline phosphatase of tobacco mosaic virus. The collection of the cDNAs can also be performed by treating the hybrids carrying DNAs corresponding to full-lengths of mRNAs with an RNase capable of cleaving DNA-RNA hybrids. As such the RNase capable of cleaving DNA-RNA hybrids, for example, RNase H can be mentioned.

A full-length coding sequence cDNA library can be obtained by synthesizing the second cDNA strands using the collected first cDNA strands as templates and cloning the obtained the second cDNA strands. The synthesis of the second cDNA strands is effected by a suitable DNA polymerase, or any suitable peptide with enzymatic activity to catalyze the elongation of a second DNA strand (in the 5′ to 3′ direction) complementary to a first DNA strand acting as a template, and the presence of dATPs, dCTPs, dGTPs, and dTTPs (Okayama & Berg (1982) Mol. Cell. Biol. 2:161). Suitable DNA polymerases are Klenow Fragment of DNA Polymerase I, E. coli DNA Polymerase, T4 DNA Polymerase, or the like. The synthesis of the second strands is brought about by a self-priming of the first cDNA strand. This reaction results in the formation of a duplex cDNA with a hairpin loop. The hairpin loop is removed by treatment with T4 DNA polymerase, which results in the blunt end of the cDNA termini and the loss of a certain amount of sequence corresponding to the 5′ end of the mRNA but not the coding sequence encoded in the mRNA. Specifically, the synthesis of the second cDNA strands is carried out without ligating a first RNA or DNA oligomer to the 3′ end of the first cDNA strands (and without the need to use a second oligomer complementary to the first oligomer to act as a primer). In addition, this present invention does not require the use of any terminal nucleotide transferase.

The disadvantage of using the homopolymers is that (a) there is the formation of multiple polymers to a single first cDNA strand, (b) due to the different lengths of the homopolymers (from multiple homopolymers) a low annealing temperature is used which causes an increase in the probability of an internal primer event, (c), when poly-G or poly-C is used, sequencing is inefficient because the sequencing enzyme more easily falls off stretches of poly-G or poly-C, and (d) the high GC content inhibits expression of the encoded peptide, because the ribosome more easily falls off high GC content stretches.

The advantage of self-priming of the first cDNA strand, instead of adding any RNA or DNA oligomer using a terminal nucleotidyl transferase, is that (a) there is a higher percent (i.e., higher efficiency) of full-length coding sequence cDNA produced because the low temperature (about 14-16° C.) used prevents internal annealing (i.e., annealing of the second primer oligo internal to the first cDNA strands), (b) there is no second primer involved, since priming has to come from the free end of the first strand of cDNA) (c) there is a higher percent of successful sequencing (for the same reason as (a) above), and/or (d) the fidelity of Taq DNA polymerase (or any other high temperature DNA polymerase), needed for the homopolymer method of synthesizing the second cDNA strands, is lower than the fidelity of DNA polymerases that can be used for the synthesis of the second cDNA strands of this present invention, which can be used at low temperature (about 14-16° C.).

In addition, steps (5) and (6) can be combined into a single step whereby addition of RNaseH (to remove separate the first cDNA strand from the beads-biotin-cap, and to remove the mRNA) and synthesis of the second cDNA strand can be carried at the same time; thereby saving an extra step.

According to the present invention, full-length cDNAs can be efficiently selected by chemically modifying the 5′ Cap site of mRNA. This is advantageous because low background and extremely high efficiency can be obtained due to the fact that the modification for the recognition of the 5′ Cap site does not depend on enzymatic reactions at all but depends on the chemical reactions utilizing the diol residue specific for the structure of the 5′ Cap site of mRNA. (In addition, there is no need to prepare recombinant CAP binding protein).

In the method of the present invention, the collection of full-length cDNAs can be performed in a solid phase system utilizing RNase I treatment and biotin-avidin reaction, which can show high selection specificity. Therefore, the method enables the production of libraries by mass productive robotics.

Step (7) can comprise the ligation of a sticky-end linker to both ends of the double stranded cDNAs. The linker, on ligation, can be given to a sequence recognized and cleavable by a restriction enzyme or not (such as recombination sequences and/or restriction sites for downstream cloning purpose). The sticky-end can be at least two to six bases long. Examples of suitable restriction enzymes are BamHI, EcoRI, SalI, or the like. In one embodiment the initial polyT primer contains at each 5′ end a sequence of a restriction site (of a first restriction enzyme that cleaves to create a sticky end), so that the linker ligated at step (7) contains sticky ends which are created by cleavage by a second restriction enzyme that creates sticky ends different from that of the first restriction enzyme. See FIG. 3 for an example. The advantage of this embodiment is that when the cDNA inserts are cleaved by both the first and second restriction enzymes, each ends of the DNA fragments produced have different sticky ends. This facilitates the directional cloning of the DNA fragments.

Step (8) comprises ligating the double stranded cDNA fragments (with sticky or blunt ends) into appropriate cloning vectors. The cloning vectors are amplifiable in a host cell. The host cell can be prokaryotic or eukaryotic. Prokaryotic host cells include bacteria, such as E. coli. Eukaryotic host cells include unicellular eukaryotic organisms, such as yeast, such as Saccharomyces cerevisiae.

In addition, for step (2), the synthesis of the first cDNA strands using reverse transcription, one can add one or a combination of saccharides, polyalcohols and chaperone proteins. Examples of suitable saccharides are trehalose, maltose, glucose, sucrose, lactose, xylobiose, agarobiose, cellobiose, levanbiose, quitobiose, 2-β-glucuronosylglucuronic acid, allose, altrose, galactose, gulose, idose, mannose, talose, sorbitol, levulose, xylitol, and arabitol. Preferably, the suitable saccharide is trehalose, sorbitol, levulose, xylitol or arabitol. Examples of suitable chaperone proteins are thermophilic bacteria chaperone proteins and heat shock proteins. Preferably, the reverse transcription is performed in the presence of one or more substances exhibiting chaperone function selected from the group consisting of saccharides and chaperone proteins. Also preferably, the reverse transcription is performed in the presence of metal ions necessary for activation of the reverse transcriptase and a chelating agent for the metal ions. Preferably, the metal ions are magnesium ions or manganese ions. Preferably, the chelating agent is one or more of deoxynucleotide triphosphates. The use of these molecules, saccharides, polyalcohols and chaperone proteins help prevent or reduce formation of secondary structures by the mRNA during reverse transcription by allowing the reverse transcription reaction to take at a higher temperature than normally allowable for the reverse transcriptase used by increasing the stability of the reverse transcriptase at the higher temperature. This increases the probability of the complete transcription over the entire transcription unit, especially the full-length of the coding sequences.

Examples of the substance exhibiting chaperone function include, but are not limited to, saccharides, amino acids, polyalcohols and their derivatives, and chaperone proteins. The “chaperone function” means a function for renaturing proteins denatured by stress such as heat shock, or a function for preventing complete denaturation of proteins by heat to maintain the native structure.

Examples of the saccharide exhibiting the chaperone function include, but are not limited to, oligosaccharides and monosaccharides such as trehalose, maltose, glucose, sucrose, lactose, xylobiose, agarobiose, cellobiose, levanbiose, quitobiose, 2-β-glucuronosylglucuronic acid, allose, altrose, galactose, gulose, idose, mannose, talose, sorbitol, levulose, xylitol and arabitol. Among these, trehalose, sorbitol, xylitol, levulose and arabitol exhibit strong chaperone function and marked effect for activating enzymes at an elevated temperature. These saccharides can be used alone or in any combination thereof.

Examples of the amino acids and derivatives thereof include, but are not limited to, N^{e}-acetyl-β-lysine, alanine, γ-aminobutyric acid, betain, N^{α}-carbamoyl-L-glutamine 1-amide, choline, dimethylthetine, ecotine (1,4,5,6-tetrahydro-2-methyl-4-pyrimidine carboxilic acid), glutamate, β-glutammine, glycine, octopine, proline, sarcosine, taurine and trimethylamine N-oxide (TMAO). Among these, betain and sarcosine exhibit strong chaperone function and marked effect for activating enzymes at an elevated temperature. These amino acids can be used alone or in any combination thereof.

Other examples of polyalcohols (since saccharides are polyalcohols) include glycerol, ethylene glycol, polyethylene glycol and the like. These polyalcohols can be used alone or in any combination thereof.

Examples of the chaperone proteins include chaperone proteins of Thermophiric bacteria and heat shock proteins such as HSP 90, HSP 70 and HSP 60. These chaperone proteins can be used alone or in any combination thereof.

These substances exhibiting chaperone function show different optimum concentrations for stabilizing the enzyme depending on the kind of the enzyme and the optimum concentration may vary among the substances for the same enzyme. Therefore, a concentration of particular substance to be added to a specific reaction system may be suitably decided depending on the kinds of the substance and the enzyme such as reverse transcriptase.

To enhance the effect of the substances exhibiting chaperone function such as saccharides, amino acids or chaperone proteins, one or more kinds of polyalcohols may be used in addition to one ore more kinds of the above substances. Examples of the polyalcohol include glycerol, ethylene glycol, polyethylene glycol and the like.

A heat-resistant reverse transcriptase is a reverse transcriptase having an optimum temperature of about 40° C. or more. Examples of heat-resistant reverse transcriptases include Tth polymerase, but the heat-resistant reverse transcriptase is not limited to this. Tth polymerase shows an optimum temperature of 70° C. and can catalyze the reverse transcription with a high activity in the above temperature range of 45° C. or higher.

When the reverse transcription is performed in the presence of the metal ions necessary for activating the reverse transcriptase, a chelating agent for the metal ions is used simultaneously. Enzymes may require metal ions for their activation. For example, Superscript II, which is a reverse transcriptase, requires magnesium ions for its activation. However, in a buffer containing magnesium ions such as a Tris buffer, fragmentation of mRNAs may proceed under the temperature condition mentioned above and hence it is difficult to obtain full length cDNAs. Likewise, Tth polymerase requires manganese ions as metal ions for its activation. However, also in a buffer containing manganese ions such as a Tris buffer, fragmentation of mRNA may actively proceed under the temperature condition as mentioned above and hence it is difficult to obtain fall length cDNAs.

To solve this problem, according to the method of the present invention, a chelating agent for metal ions is added to the system so that the activity of reverse transcriptase should be maintained and the fragmentation of mRNAs can be prevented. However, if all of the metal ions necessary for the activation of the reverse transcriptase are chelated, the reverse transcriptase loses its activity. Therefore, it is suitable to use a chelating agent of comparatively weak chelating power.

Examples of such a chelating agent of comparatively weak chelating power include deoxynucleotide triphosphates (dNTPs). The chelating agent of comparatively weak chelating power is suitably used in an approximately equimolar amount of the metal ion. When a deoxynucleotide triphosphate is used as the chelating agent, for example, it is suitable to add an approximately equimolar amount of deoxynucleotide triphosphate as to the metal ion. Accordingly, the amount of the chelating agent can be suitably decided with consideration to the chelating power as to the objective metal ion, so that the reverse transcriptase activity can be maintained and the fragmentation of mRNAs can be prevented. The deoxynucleotide triphosphates, dATP, dGTP, dCTP and dTTP, may be used alone or in any combination thereof All of the four kinds of dNTPs, dATP, dGTP, dCTP and dTTP, may be used together. Since these can serve also as substrates of the reverse transcription, all of them are usually used together.

The following examples further illustrate the present invention. These examples are intended merely to be illustrative of the present invention and are not to be construed as being limiting.

### EXAMPLES

### Example 1

CAP-Capture cDNA Synthesis to Generate Full-Length Coding Sequence cDNA.

First strand synthesis. Begin with 10 μg of pelleted mRNA per reaction. Resuspend each pelleted mRNA in 16 μL of NotI primer-adapter per reaction. The reaction is then heated. When the reaction mixture reaches 45° C., add, to each reaction, 20 μL of 5× first -strand buffer (Gibco), 10 μL 0.1 M DTT (Gibco), 1 μL RNase Inhibitor (Roche), 37 μL preheated saturated trehalose, 5 μL 10 mM dNTPs (Gibco), 1 μL 100×BSA (New England Biolab), and 10 μL ImProm-II (Promega).

The reaction mixture then heated to 55°. From 55° the reaction mixture is heated to 60° at the rate of an increase of half a degree for every 1 minute. The reaction mixture is incubated at 60° C. for 2 minutes, followed by incubation at 55° C. for 2 minutes for one cycle. Ten such cycles are carried out followed by being held at 4° C.

Then to each reaction mixture is added 2 μL 20 mg/mL Proteinase K (Gibco). Incubate at 45° C. for 15 minutes. To each reaction mixture add 10 μL 3 M NaOAc (pH 6, DEPC-treated), and 250 μL of 100% ethanol or 100 μL of 100% isopropanol. Freeze at −80° C. for 30 minutes or more. Centrifuge at 14,000 rpm at 4° C. for 30 minutes. Wash pellet with 70% (v/v) ethanol/30% DEPC-treated dd H₂O. Centrifuge at 14,000 rpm at 4° C. for 30 minutes. Remove excess supernatent, air dry pellet at 37° C. for 10 minutes.

Biotinylation. Resuspend pellet in 85 μL DEPC-treated dd H₂O. Add 10 μL 1 M NaOAc (pH 4.5, DEPC-treated dd H₂O), and 5 μL freshly prepared 100 mM (21.4 mg/mL) NaIO₄ (ICN). Mix and incubate on ice in the dark for 45 minutes. Then add 1 μL 10% (w/v) SDS, 122 μL 100% isopropanol, and 22 μL 5 M RNase-free NaCl (Ambion). Mix and incubate on ice in the dark for 30 minutes. Centrifuge at 14,000 rpm at 4° C. for 30 minutes. Wash pellet with 70% (v/v) ethanol/30% DEPC-treated dd H2O. Centrifuge at 14,000 rpm at 4° C. for 30 minutes. Remove excess supernatent, air dry pellet at 37° C. for 10 minutes. Resuspend pellet in 50 μL DEPC-treated dd H₂O. Then add 5 μL 1 M NaOAc (pH 6.1, DEPC-treated dd H₂O), 150 μL 10 mM (4.8 mg/mL) long-arm biotin hydrazide (Molecular Probes). Incubate at room temperature in the dark overnight.

Purification of full-length RNA-DNA heteroduplex. To each reaction mixture add 5 μL 5 M NaCl, 75 μL 1 M RNase-free NaOAc (pH 6.1, DEPC-treated dd H₂O), and 750 μL of 100% ethanol or 100 μL of 100% isopropanol. Freeze at −80° C. for 30 minutes or more. Centrifuge at 14,000 rpm at 4° C. for 30 minutes. Wash pellet with 70% (v/v) ethanol/30% DEPC-treated dd H₂O. Centrifuge at 14,000 rpm at 4° C. for 30 minutes. Remove excess supernatent, air dry pellet at 37° C. for 10 minutes. Resuspend pellet in 80 μL DEPC-treated dd H2O. Then add 10μ 10× RNaseI buffer (1 unit RNAseI/μg of starting mRNA (Promega)). Incubate at 37° C. for 15 minutes or more.

Then add 2.5 μL of 40 mg/mL yeast tRNA. While the biotinylated RNA/DNA heteroduplx is precipitating, prepare the streptavidin-labelled Dynabeads. Pippette 400 μL of M-280 Streptavidin beads (Dynal) into an RNase-free Eppendorf tube. Place on magnet, wait for at least 30 seconds and remove supernatent. Resuspend beads in 400 μL 1× binding buffer (2 M NaCl, 50 mM EDTA). Place on magnet, wait for at least 30 seconds and remove supernatent. Resuspend beads in 400 μL 1× binding buffer (2 M NaCl, 50 mM EDTA) with 50 μg/mL yeast tRNA. Incubate at room temperature with rotation for 30 minutes or more. Place on magnet, wait for at least 30 seconds and remove supernatent. Resuspend beads in 100 μL 2× binding buffer.

Mix beads and RNA-DNA heteroduplex. Incubate at room temperature with rotation for 30 minutes or more. Place on magnet, wait for at least 30 seconds and remove supernatent. Wash twice with 400 μL of 1× binding buffer. Place on magnet, wait for at least 30 seconds and remove supernatent. Wash with 400 μL of 0.4% (w/v) SDS plus 50 μg/mL yeast tRNA. Place on magnet, wait for at least 30 seconds and remove supernatent. Wash with 400 μL of 1× wash buffer (10 mM Tris-HCL pH 7.5, 0.2 mM EDTA, 10 mM NaCl, 20% (v/v) glycerol, 50 μg/nL yeast tRNA. Place on magnet, wait for at least 30 seconds and remove supernatent. Wash with 400 μL of 50 μg/mL yeast tRNA. Place on magnet, wait for at least 30 seconds and remove supernatent. Wash with 400 μL of 1× second strand buffer (Gibco cDNA synthesis kit). Place on magnet, wait for at least 30 seconds and remove supernatent.

Second strand synthesis. Resuspend in 72 μL DEPC-dd H₂O. Then add 20 μL 5× second strand buffer, 4 μL E. coil DNA polymerase, 2 μL 10 mM dNTP, 1 μL E. coli RNaseH, and 1 μL E. coli ligase. Incubate at 16° C. for two hours or more (lightly resuspend beads every 15 minutes for the first 30 minutes). Add 2 μL T4 DNA polymerase. Incubate at 16° C. for five minutes. Add 10 μL 0.5 M EDTA and mix. Place on magnet, wait for at least 30 seconds and remove supernatent (save supernatent). Wash beads once with 100 μL dd H₂O. Place on magnet, wait for at least 30 seconds and remove supernatent (save supernatent). Add 200 μL phenol:CHCl₃:IAA. Flick tube to mix. Centrifuge at 14,000 rpm at room temperature for 2 minutes. Remove and save the supernatent. Re-extract phenol:CHCl₃:IAA with 100 or 200 μL fresh dd H₂O. Pool aqueous phases.

Add 0.1 volume of 3 M NaOAc (pH 6.0) and 2 to 2.5 volume 100% ethanol. Freeze at −80° C. for 30 minutes or more. Centrifuge at 14,000 rpm at 0° C. for 30 minutes. Wash pellet with 70% (v/v) ethanol/30% DEPC-treated dd H₂O (RNase-free). Centrifuge at 14,000 rpm at 0° C. for 10 minutes. Remove excess supernatent, air dry pellet at room temperature for 15 minutes. Resuspend pellet in 25 μL dd H₂O. Place on ice, and add 10 μL 5× T4 ligase buffer, 10 μL SalI adaptor, and 5 μL T4 ligase (Gibco cDNA synthesis kit). Incubate at 16° C. overnight.

Final preparations for cloning. Add 0.1 volume 3 M NaOAc (pH 6.0) and 2 to 2.5 volume 100% ethanol or 1 volume isopropanol. Freeze at −80° C. for 30 minutes or more. Centrifuge at 14,000 rpm at 0° C. for 30 minutes. Wash pellet with 70% (v/v) ethanol/30% DEPC-treated dd H₂O (RNase-free). Centrifuige at 14,000 rpm at 0° C. for 10 minutes. Remove excess supernatent, air dry pellet at room temperature for 15 minutes or 5 to 10 minutes at 37° C. Put the reaction mixture on ice and add q.s. to 41 μL with dd H₂O. Then add 5 μL buffer 3. Then add 4 μL NotI restriction enzyme. Incubate at 37° C. for one to two hours. Q.s. to 105 μL with T₁₀E_{0.1}N₂₅ buffer for the sizing column. Reserve 5 μL for gel and spectrometer analysis.

Prepare cDNA size fractionation columns (Gibco) by uncapping the column (bottom first) and allowing it to drain completely. Wash with 5×800 μL T₁₀E_{0.1}N₂₅ buffer by allowing the column to drain completely each time. Load the DNA sample onto the column. Collect the flow-through in an Eppendorf tube. Add 100 μL T₁₀E_{0.1}N₂₅ buffer. Collect the flow-through, 1 drop per pre-numbered Eppendorf tube. Whenever the column runs dry, add another 100 μL T₁₀E_{0.1}N₂₅ buffer. According to the protocol of Gibco, up to drop 20 is to be collected. Drops 12-20 should also be pooled. Use 4 μL from each fraction to visualize on a gel. Pool fractions showing cDNA≧1.0 kbp (up to 2-3 kbp). Measure the samples with a spectrometer. (Use a cuvette soaked at least 30 minutes in slightly acidified 100% ethanol. Rinse five times with dd H₂O.) Save each sample. Samples identified containing cDNA from 1.0 to 3.0 kbp can then be ligated into a suitable amplifiable vector. If only one fraction is used, precipitate it and use from half to all of the cDNA in it.

Although the invention has been described with reference to the presently preferred embodiments, it should be understood that various modifications can be made without departing from the spirit of the invention. All publications, patents, and patent applications are herein incorporated by reference in their entirety to the same extent as if each individual patent, or patent application was specifically and individually indicated to be incorporated by reference in its entirety.

1. A method for making a full-length coding sequence cDNA library, comprising:
(a) forming RNA-DNA hybrids by reverse transcription starting from primers using mRNAs as templates;
(b) binding a tag molecule to a diol structure present in the 5′ cap site of a mRNA forming a RNA-DNA hybrid; and
(c) separating RNA-DNA hybrids carrying a DNA corresponding to a full-length mRNA from the RNA-DNA hybrids formed above by binding the tag molecule, wherein said DNA corresponding to a full-length mRNA are first cDNA strands;
wherein said full-length coding sequence cDNA library is a library of cDNAs comprising the full-length of the coding sequences and having lengths less than the full-length of mRNAs.

2. The method according to claim 1, further comprising the step of synthesizing second cDNA strands using as templates said first cDNA strands, wherein ligating an RNA or DNA oligomer to the 3′ end of said first cDNA strands is not required.

3. The method according to claim 2, said synthesizing comprises self-priming said first cDNA strand.

4. The method according to claim 1, further comprising the step of digesting RNA-DNA hybrids binding tag molecules with an RNase capable of cleaving single strand RNA to cleave the single strand RNA parts of the RNA-DNA hybrids carrying a DNA not corresponding to a full-length mRNA to remove tag molecules from the hybrids.

5. The method according to claim 4, wherein said synthesizing and said digesting at least overlap.

6. The method according to claim 4, wherein said RNase capable of cleaving single stranded RNA is ribonuclease I.

7. The method according to claim 1, wherein the primer is oligo dT.

8. The method according to claim 1, wherein the diol structure present in the 5′ cap site of the mRNA is subjected to a ring-open reaction by oxidation with periodic acid to form a dialdehyde and the dialdehyde is reacted with a tag molecule having a hydrazine terminus to form a mRNA binding the tag molecule.

9. The method according to claim 8, wherein the tag molecule having a hydrazine terminus is a biotin molecule having a hydrazine terminus or an avidin or streptavidin molecule having hydrazine terminus.

10. The method according to claim 1, wherein the tag molecule is a biotin molecule having a functional group which is capable of binding a diol structure present in the 5′ cap site of mRNA, and the hybrids carrying a DNA corresponding to a full-length mRNA are separated by utilizing binding properties of avidin or streptavidin fixed on a solid support to the biotin molecule which is the tag molecule of the RNA-DNA hybrid.

11. The method according to claim 1, wherein the tag molecule is an avidin or streptavidin molecule having a functional group which is capable of binding a diol structure present in the 5′ cap site of mRNA, and the hybrids carrying a DNA corresponding to a full-length mRNA are separated by utilizing binding properties of biotin fixed on a solid support to the avidin or streptavidin molecule which is the tag molecule of the RNA-DNA hybrid.

12. A method for constructing a full-length coding sequence cDNA library, comprising:
(a) binding a tag molecule to a diol structure present in 5′ cap sites of mRNAs by oxidizing the 5′ cap site diol to form a dialdehyde and reacting the resulting dialdehyde with a tag molecule having a group reactive with the dialdehyde;
(b) forming RNA-DNA hybrids by reverse transcription using primers and the mRNAs binding the tag molecule as templates; and
(c) separating RNA-DNA hybrids carrying a DNA corresponding to a full-length of mRNA from the RNA-DNA hybrids formed above by using a function of the tag molecule, wherein said DNA corresponding to a full-length mRNA are first cDNA strands;
wherein said full-length coding sequence cDNA library is a library of cDNAs comprising the full-length of the coding sequences and having lengths less than the full-length of mRNAs.

13. The method according to claim 12, further comprising the step of synthesizing second cDNA strands using as templates said first cDNA strands, wherein ligating an RNA or DNA oligomer to the 3′ end of said first cDNA strands is not required.

14. The method according to claim 13, said synthesizing comprises self-priming said first cDNA strand.

15. The method according to claim 12, further comprising the step of digesting RNA-DNA hybrids binding tag molecules with an RNase capable of cleaving single strand RNA to cleave the single strand RNA parts of the RNA-DNA hybrids carrying a DNA not corresponding to a full-length mRNA to remove tag molecules from the hybrids.

16. The method according to claim 15, wherein said synthesizing and said digesting at least overlap.

17. The method according to claim 12, wherein the primer is oligo dT.

18. The method according to claim 12, wherein the tag molecule is a biotin molecule having a functional group capable of binding to a diol structure present in 5′ cap site of mRNA and the RNA-DNA hybrids carrying a DNA corresponding to a full-length of mRNAs are separated by utilizing binding between an avidin molecule fixed on a solid support and a biotin molecule possessed by the RNA-DNA hybrids as the tag molecule.

19. The method according to claim 12, wherein the tag molecule is an avidin molecule having a functional group capable of binding to a diol structure present in 5′ cap site of mRNA and the RNA-DNA hybrids carrying a DNA corresponding to a full-length of mRNAs are separated by utilizing binding between a biotin molecule fixed on a solid support and an avidin molecule possessed by the RNA-DNA hybrids as the tag molecule.

20. The method according to claim 12, wherein the diol structure present in 5′ Cap site of mRNA is subjected to a ring-open reaction by oxidation with sodium periodate to form a dialdehyde and the dialdehyde is reacted with a tag molecule having a hydrazine terminus to form mRNA binding the tag molecule.

21. The method according to claim 20, wherein the tag molecule having a hydrazine terminus is a biotin molecule or avidin molecule having a hydrazine terminus.

22. The method according to claim 12, wherein the RNA-DNA hybrids are digested with an RNase capable of cleaving single strand RNA to cleave the single strand parts of the hybrids so that the tag molecule is removed from those hybrids carrying a DNA not corresponding to a full-length mRNAs and then those hybrids carrying a tag molecule and a DNA corresponding to a full-length of mRNAs are separated.

23. The method according to claim 22, wherein the RNase capable of cleaving single strand RNA is ribonuclease I.

24. A method for making a full-length cDNA library, comprising:
(a) binding a biotin molecule to a diol structure present in 5′ cap sites of mRNAs by oxidizing the 5′ cap site diol to form a dialdehyde and reacting the resulting dialdehyde with a biotin molecule having a group reactive with the dialdehyde;
(b) forming RNA-DNA hybrids by reverse transcription using primers and the mRNAs bound to biotin molecules as templates;
(c) digesting the RNA-DNA hybrids with an RNase capable of cleaving single strand RNA to cleave the single strand RNA parts of the hybrids carrying a DNA not corresponding to a full-length mRNA to remove biotin molecules from the hybrids, wherein said DNA not corresponding to a full-length mRNA is a first cDNA strand; and
(d) separating RNA-DNA hybrids carrying a DNA corresponding to a full-length mRNA and binding the biotin molecules by (1) allowing them to react with avidin fixed on a solid support or (2) affinity chromatography to a solid support;
wherein said full-length coding sequence cDNA library is a library of cDNAs comprising the full-length of the coding sequences and having lengths less than the full-length of mRNAs.

25. The method according to claim 24, further comprising the step of synthesizing second cDNA strands using as templates said first cDNA strands, wherein ligating an RNA or DNA oligomer to the 3′ end of said first cDNA strands is not required.

26. The method according to claim 25, said synthesizing comprises self-priming said first cDNA strand.

27. The method according to claim 26, wherein said synthesizing and said digesting at least overlap.

28. The method according to claim 27, wherein the primer is oligo dT and the RNase capable of cleaving single strand RNA is ribonuclease I.

29. A method for making a fall-length cDNA library, comprising:
(a) binding an avidin molecule to a diol structure present in 5′ cap sites of mRNAs by oxidizing the 5′ cap site diol to form a dialdehyde and reacting the resulting dialdehyde with an avidin molecule having a group reactive with the dialdehyde;
(b) forming RNA-DNA hybrids by reverse transcription using primers and the mRNAs bound to avidin molecules as templates;
(c) digesting the RNA-DNA hybrids with an RNase capable of cleaving single strand RNA to cleave the single strand RNA parts of the hybrids carrying a DNA not corresponding to full-length mRNAs to remove avidin molecules from the hybrids, wherein said DNA not corresponding to a full-length mRNA is a first cDNA strand; and
(d) separating RNA-DNA hybrids carrying a DNA corresponding to a full-length mRNA and binding avidin molecules by (1) allowing them to react with biotin fixed on a solid support or (2) affinity chromatography to a solid support;
wherein said full-length coding sequence cDNA library is a library of cDNAs comprising the full-length of the coding sequences and having lengths less than the full-length of mRNAs.

30. The method according to claim 29, further comprising the step of synthesizing second cDNA strands using as templates said first cDNA strands, wherein ligating an RNA or DNA oligomer to the 3′ end of said first cDNA strands is not required.

31. The method according to claim 30, said synthesizing comprises self-priming said first cDNA strand.

32. The method according to claim 31, wherein said synthesizing and said digesting at least overlap.

33. The method according to claim 32, wherein the primer is oligo dT and the RNase capable of cleaving single strand RNA is ribonuclease I.
